(19) United States
(12) Patent Application Publication
(10) Pub. No.: US 2020/0138750 A1
(43) Pub. Date: May 7, 2020

(54) Use of Mustard Seed Oil (Allyl Isothiocyanate) to relieve headache pain

(71) Applicants: Ami Snehal Panchal, Tuscaloosa, AL (US); Snehal B. Panchal, Tuscaloosa, AL (US)

(72) Inventors: Ami Snehal Panchal, Tuscaloosa, AL (US); Snehal B. Panchal, Tuscaloosa, AL (US)

(21) Appl. No.: 16/555,554

(22) Filed: Aug. 29, 2019

Related U.S. Application Data
(60) Provisional application No. 62/726,392, filed on Sep. 3, 2018.

(51) Int. Cl. A61K 31/145

(52) U.S. Cl. CPC A61K 31/145

(57) ABSTRACT

An extract derived from a natural component is used to treat headaches. Natural component is known as Mustard Seeds and the extract is known as Allyl Isothiocyanate (AITC). AITC has anti-inflammatory, anti-microbial and anti-coagulative properties. This natural product or extract is deliverable as a nasal via inhale method. The Potent, Pungent and counterirritant properties of AITC vapor is the cure of headaches pain.
Headaches can be described as any of this but not limited to tension headaches, cluster headaches, migraines allergy or sinus headaches, hormone headaches, caffeine headaches, exertion headaches, hypertension headaches, rebound headaches, post-traumatic headaches etc.

### FIELD OF INVENTION

The field of invention relates to ancient and traditional natural therapy used in Ayurveda science aka Ayurvedic medicine.

### BACKGROUND OF THE INVENTION

The present invention relates to relieve and treat headaches (defined as a continuous pain in the head) caused by several chronic and acute reasons but not limited to Migraine, stress, blood pressure, toothache, allergies, lack of sleep, Tension etc.

Headache is the symptom of pain anywhere in the region of the head or neck. Headaches may occur on one or both sides of the head, be isolated to a certain location, radiate across the head from one point, or have a viselike quality.

A headache may appear as a sharp pain, a throbbing sensation or a dull ache. Headaches can develop gradually or suddenly, and may last from less than an hour to several days.

It occurs in migraines (sharp, or throbbing pains), tension-type headaches, and cluster headaches. Frequent headaches can affect relationships and employment. There is also an increased risk of depression in those with severe headaches.

Headaches can occur as a result of many conditions whether serious or not. There are a number of different classification systems for headaches. Causes of headaches may include dehydration, fatigue, sleep deprivation, stress, the effects of medications, the effects of recreational drugs, viral infections, loud noises, common colds, head injury, rapid ingestion of a very cold food or beverage, and dental or sinus issues.

Treatment of a headache depends on the underlying cause, but commonly involves pain medication. A headache is one of the most commonly experienced of all physical discomforts.

About half of adults have a headache in a given year. Tension headaches are the most common, affecting about 1.6 billion people (21.8% of the population) followed by migraine headaches which affect about 848 million (11.7%)

Many treatments for the headaches currently exist. These treatments may be obtained over the counter or with a prescription and include, without limitation, anti-inflammatory, pain-relieving over-the-counter medicines, antihistamines, decongestants, NSAID, steroids, etc. Treatments may come in various different forms such as, but not limited to, pills, liquids or nasal sprays.

At the onset of pain, most people reach for either an acetaminophen (Tylenol) or a nonsteroidal anti-inflammatory drug (Aspirin, Advil, Aleve), also known as NSAIDs, which can be very effective in fighting pain. In fact, these medications are recommended in the CDC's new guidelines, which say, “In particular, acetaminophen and NSAIDs can be useful for arthritis and low back pain.” Traditional over-the-counter and prescription headaches medications offer only short-term benefits and may cause side effects including, some sinus causing headaches treatments involve the use of steroids; however, these treatments may irritate the nasal passages or cause bleeding. Furthermore, steroidal or medicated treatments may harmfully interact with other medications and may be addictive

Some available headache treatments comprise caffeine and some low to moderate doses of narcotics. Both of these contain are proven to have severe side effects, misuse of them causes rebound headaches. And overuse might cause possible addiction as well.

About 25 percent of over-the-counter painkillers are used by buyers to treat headaches, some patients overmedicate in an attempt to quickly rid their pain, but taking more than a recommended dose may not resolve a headache. In fact, it may only make matters worse, as the National Headache Foundation says three or more doses of painkillers per day for five days can sometimes lead to rebound headaches.

All of the above currently available treatment for headache takes at least 30 minutes to get effective and none are proven to cure the headache instead they only suppress the headache pain.

Several chronic Migraine headache patience go for vigorous and expensive Migraine clinics and even surgery to spend hours and hours into treating headache for years.

In view of the foregoing, there is a need for improved techniques for providing an Ayurvedic treatment for acute or chronic conditions headaches that is non-habit forming and does not cause rebound headaches.

The objective of the present invention provides a treatment for headaches that does not use chemicals, antibiotics, steroids, caffeine, narcotics (Opioid) or other conventional medications chemicals.

### SUMMARY OF THE INVENTION

The attempt of this invention is to provide Natural, Herbal, Organic yet practically instant and quickest remedy for headache relief.

The present embodiment focuses the use of Mustard Seed Oil known as Allyl Isothiocyanate or AITC as acronym produced from Mustard seeds to relieve headache pain naturally is described and experienced on users with headaches.

The use of Allyl Isothiocyanate (Mustard Seed oil or extract) in this embodiments, further describe is very limited to quick inhale method and not intended to ingest or consumed, or applied topically, which makes it even more consumer friendly and convenient method of treating headaches compared to available conventional other pain treatments.

Allyl isothiocyanate is obtained from the seeds of black mustard (Brassica nigra) or brown Indian mustard (Brassica juncea). When these mustard seeds are broken, the enzyme myrosinase is released and acts on a glucosinolate known as sinigrin to give allyl isothiocyanate.

In present embodiment, Mustard Seed oil is referred as Allyl Isothiocyante or AITC.

Allyl isothiocyanate is produced commercially by the reaction of allyl chloride and potassium thiocyanate.
CH2−CHCH2Cl+KSCN→CH2=CHCH2NCS+KCl
Allyl Isothiocyanate is identifier as—CAS-57-06-7
Chemical name/formula—C4H5NS

The product obtained in this fashion is sometimes known as synthetic mustard oil. Allyl isothiocyanate can also be liberated by dry distillation of the seeds. The product obtained in this fashion is known as volatile oil of mustard and is usually around 92% pure. It is used principally as a flavoring agent in foods.

AITC is colorless oil and it is responsible for the pungent taste of mustard. This pungency and the lachrymatory effect of AITC are mediated through the TRPA1 and TRPV1 ion channels.

In the present invention AITC is taken in to its purest form. To treat headache instantly, Purest AITC needs to be inhaled deeply for up to 5 seconds in each nostril. If it is taken correctly, due to its pungency and potency, the vapor of AITC will instantly felt inside on the top of the crown of the user's head.

Users are suggested to wear eyeglasses while using this product to avoid irritations into eyes.

For those who are skilled in this art, can simply keep their eyes closed to use this product to avoid the irritations.

Further, if it is inhaled deeply, user will feel instantly irritated and most likely get tears from eyes due to its lachrymal effect. This effect is not to consider a side effect yet it is only a sign of proper inhalation of the vapor thru nostrils. The potency, pungency and irritable effects of AITC is the cure of headache instantly.

Users mostly feel very or extremely (depends on their tolerance level) irritated due to it being so strong and solid vapor, however due to the counterirritant nature of AITC, the blood circulation improves wherever the inflammation or congestion presents. It stimulates nerve endings and that relieves the pressure built within the head causing headache to go instantly.

If AITC is inhaled properly and as directed, most users will be out of irritation in less than 2 minutes. At the same time the headache is also relieved.

Inhaling AITC also, helps clearing up mucus built into the nasal passage and thus it removes pressure from sinus and relives sinusitis pressure and sinus headaches.

Therefore, it can also be used when user is suffering from common cold, allergies or any such upper respiratory symptoms where congestion is involved along with headaches.

After use of AITC, mucus may start coming out from nose or by mouth, it is a sign of clearing up the sinuses and nasal passages. It will be varying by user the amount and frequency of mucus discharge.

If Pure AITC (Allyl Isothiocyanate) inhaled as directed, most users suffering with headaches will feel relived from pain in seconds in most cases or up to couple of minutes. The time it gives the relief within is significantly less than any other pain reliving medicines are available in market.

The fast relief from a natural product without ingestion is the main key of the invention. Such product does not exist currently and neither available for purchase or import in current market.

The users, who have tried this remedy, are either completely relieved from headaches, or have larger intervals between migraine episodes, or get less potent headaches than before.

According to Ayurveda Mustard oil has been used traditionally to treat several symptoms related to pain and blood. However, use of AITC via inhalation to relieve from headache is not conveniently available to vast users who are suffering from headaches frequently and thus, attempt to use AITC in a convenient method will help several millions people who are suffering from various types of headaches daily.

In the present embodiment, use of Mustard seed oil or AITC to treat headache in method of inhale via but not limited to vials, sachets, swabs etc. is natural, innovative and convenient approach to the users suffering from headaches.

According to Ayurvedic medicine, AITC aka Mustard Oil has been used vastly to treat various types of pain externally and it is still in use, however, it has not been so much in favor as the use of it has been limited to massage externally, ingesting the juice or seeds orally or applied power or paste form of Mustard topically etc.

Above methods of using this potential medicine is not very convenient and readily available anywhere or favorable to users in emergency headache episodes. Present embodiment provides this potential medicine via very innovative, productive and yet very effective way.

Mustard Oil is classified as an Oil and no user will feel comfortable to carry the oil for the headache relief.

Present embodiment focuses on how to provide the extract of Mustard Oil aka AITC to users easily and in favorable packing to use in emergency.

This invention is very convenient approach for the people who suffer from frequent headaches and those who are reluctant to constant use of OTC pain killers or prescriptions drugs.

In this invention AITC is used in its purest and fresh form. It can also be altered with mixing any other kind of organic material but not limited to essential oils like, lavender, tea tree, camphor, eucalyptus etc. to mask the pungent smell (not to override) and make it more user friendly. It can also be mixed with above but not limited to essential oils to get inclined benefit of calming and soothing due to several other side effects of cold, allergy, tension, stress etc.

In the present embodiment, the AITC remedy does not have any side effects, is non-habit forming, non-drowsy. The solution provides hygienic headache relief. The use of the present embodiment and simple, daily inhalation provide a lifetime of migraine relief and comfort. Unlike many headache remedies, using AITC according to the present embodiment does not lead to addiction or rebound headaches. The solution is non-steroidal, does not promote antibiotic resistance, and has no known drug interactions. The solution is safe for many users including, without limitation, adults, children 15 years and older, diabetics, pregnant or lactating women, hypertensive individuals, etc.

If using pure AITC, the quantity of AITC is 0.1 ml to 2 ml in a single pack. With that it can have its highest potency and pungency. If it is less, it may not be as effective as it is in a larger volume.

In the present embodiment, quantity of AITC and carrier of AITC in which AITC is stored and used by users to treat their headaches is only for estimation and trials.

Final product of AITC and the quantity and packing of the final product of AITC is still being developed and it not finalized.

Studies are being performed on how much AITC is enough to treat headache instantly as described above.

Studies are also being performed on storing and shelving life of AITC for its most effectiveness to treat headaches.

In current studies we have used pure AITC in above mentioned quantity into small dark thick glass vials with aluminum seal and tight screw caps. The bottles are also very small mouth.

Present embodiment also focuses on variety of possible approaches to provide AITC to its users in a convenient way. Which are like but not limited to, glass vials, cotton swab sachets, pads, pouches with foam absorbing AITC, inhaling tubes etc.

### BRIEF DESCRIPTION OF DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 Shows user is demonstrated to inhale Allyl Isothiocyanate—AITC (stored in inhaler tube) via nose by one nostril. User is supposed to inhale AITC from both of the nostrils for up to 5 seconds. User is suggested to keep eyes closed while inhaling.

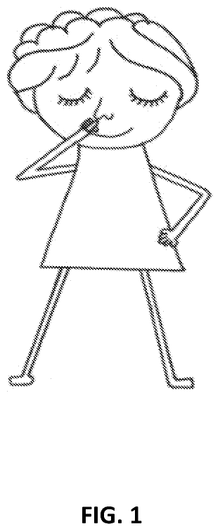

FIG. 2 Shows user is demonstrated to inhale Allyl Isothiocyanate—AITC (stored in inhaler tube) via nose by another nostril. User is supposed to inhale AITC from both of the nostrils for up to 5 seconds. User is suggested to keep eyes closed while inhaling.

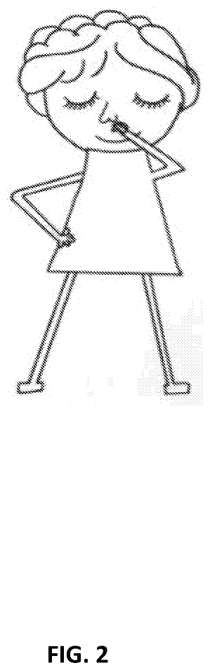

FIG. 3 Shows various ways user can inhale Allyl Isothiocyante (AITC) for example like Inhaler tube, use and throw sachet, small vials etc. These method of inhaling AITC is few examples of several other ways user can inhale AITC during headache pains.

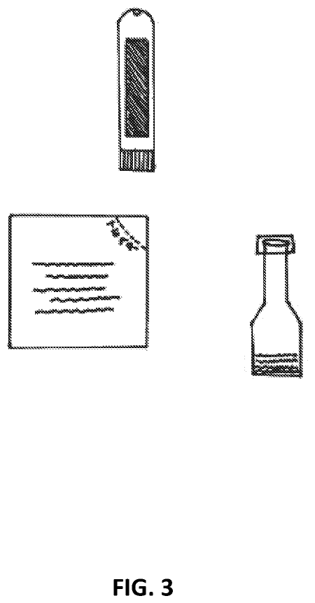

### OTHER RESOURCES OF OBTAINING AITC—ALLYL ISOTHIOCYANTE

Isothiocyanate is the chemical group —N═C═S, formed by substituting the oxygen in the isocyanate group with a sulfur. Many natural isothiocyanates from plants are produced by enzymatic conversion of metabolites called glucosinolates. These natural isothiocyanates, such as allyl isothiocyanate, are also known as mustard oils. An artificial isothiocyanate, phenyl isothiocyanate, is used for amino acid sequencing in the Edman degradation.

Other than Black Mustard seeds including and not limited to Brown, white, yellow or any other available kind of Mustard seeds can be used to produce AITC.

This same Natural Allyl Isothiocyanate (AITC) is responsible for the pungent taste of radish, horseradish, and wasabi. Wasabi, Mustard, Radish, Horseradish etc. are all Brassicaceae or Cruciferae family of plants, and AITC is can be produced from any of plants from this family.

Combinations of natural Isothiocyante obtained from Brassicaceae or Cruciferae family of plants can also get similar pungent, potent and lachrymatory properties available in Natural Isothiocyante, as in Allyl Isothiocyanates and can be used to treat Headaches pain.

Both Natural and synthetic AITC gives almost same result in treating headaches, however, Synthetic is made using chemical formula and naturally obtained AITC is made from Mustard seeds, Horseradish or wasabi. Due to the making of AITC varies naturally, it is not 100% pure always. Chemically or synthetically obtained AITC is 99.9% pure.

Regardless of the resource or manufacturing of AITC, headaches and relevant symptoms are treated due to its pungent, potent and lachrymatory properties. Pungency and the lachrymatory effect of AITC are mediated through the TRPA1 and TRPV1 ion channels.

I/we claim

1. Allyl Isothiocyanates or Mustard Seed Oil produced naturally which is characterized by the production of glucosinolates, and of the enzyme myrosinase, which acts on glucosinolates to release isothiocyanates is used for treating any kind of headaches.

2. Natural or synthetic isothiocyanate like but not limited to Allyl isothiocyanate is one of the main active or inactive component of any solution made out to use treat headaches.

3. The pungency and the lachrymatory effect of AITC or any other isothiocyanates are mediated through the TRPA1 and TRPV1 ion channels that helps treating headaches of any kind.

4. Allyl Isothiocyante produced via but not limited to natural, artificial, chemical reaction, synthetic, combination of natural Isothiocyante found in Brassicales family of plants or combinations of chemically produced isothiocyanates are being used for headache treatment.

5. Combinations of Natural Isothiocyanates produced and occurred widely in vegetables and are members of the Brassicales plant family and are being used for headaches.

6. Isothiocyanates produced naturally or artificially/chemically comprising irritant, pungent and potent lachrymatory properties are being used to treat headaches.

7. Natural isothiocyanates or synthetic isothiocyanate, in particular but not limited to Allyl Isothiocyante is being inhaled for headache relief.

8. Natural isothiocyanates or synthetic isothiocyanate, in particular but not limited to Allyl Isothiocyante is used to treat Headache administered via any method but not limited to inhale, mist, tablet, pills, rub, spray, lotion,solution etc.

9. Natural Allyl isothiocyanate or synthetic Allyl isothiocyanate is used to treat headache pain caused by any symptoms but not limited to Migraine, toothaches, sinus, cold, congestion, fever, allergies, blood pressure, tension, stress, cluster etc. Headaches can be described as any of this but not limited to tension headaches, cluster headaches, migraines allergy or sinus headaches, hormone headaches, caffeine headaches, exertion headaches, hypertension headaches, rebound headaches, post-traumatic headaches etc.

10. Natural Allyl isothiocyanate or synthetic Allyl isothiocyanate is derived from Wasabi, Radish or Horseradish of any kind or Mustard Seeds of any kind including but not limited to white, black or brown color.

11. Natural Allyl isothiocyanate or synthetic Allyl isothiocyanate is prepared using chemical reaction of any kind but not limited to allyl chloride and potassium thiocyanate.

12. Natural Allyl isothiocyanate or synthetic Allyl isothiocyanate comprises anti-inflammatory, anti-microbial, anti-coagulative properties.

13. Natural Allyl isothiocyanate or synthetic Allyl isothiocyanate provided to user in form but not limited to Inhaler tubes, Inhaler sticks, bottle, swab, sachet, foam pad, vials, spray, nasal, Neti-pot etc. is used to treat headaches and relevant possible symptoms like but not limited to congestion, sinus, muscles pain due to headache etc. upper respiratory dysfunctions.

14. Natural Allyl isothiocyanate or synthetic Allyl isothiocyanate mixed with any organic solvent or mixed with any other substances and used for treating pain in head, aka headaches of any kinds.
